(19) United States
(12) Patent Application Publication
(10) Pub. No.: US 2003/0073930 A1
(43) Pub. Date: Apr. 17, 2003

(54) Methods and device for preventing or healing injured nipples or areolas in mammalian females or for obtaining samples from nipples in mammalian females

(76) Inventors: Gerald R. Morrissey, Skaneateles, NY (US); Robert J. Corona JR., Skaneateles, NY (US)

Correspondence Address: Georgia Evans, NIXON PEABODY LLP, Clinton Square, P.O. Box 31051, Rochester, NY, 14603, US

(21) Appl. No.: 09/981,458

(22) Filed: Oct. 17, 2001

(51) Int. Cl. A61B 5/00; B65D 81/00; A61J 13/00

(52) U.S. Cl. 600/573; 128/890

(57) ABSTRACT

A method for preventing or healing injured nipples or areolas in mammalian females. The method involves positioning an absorbent medium over at least a portion of a nipple and areolar region of a mammalian female breast. Next, pressure is applied to the absorbent medium over at least a portion of the nipple and areolar region sufficient to prevent or heal injured nipples or areolas.

### FIELD OF THE INVENTION

The present invention relates to methods and a device for preventing or healing sore, irritated, damaged, or infected nipples or areolas in mammalian females. In addition, the present invention relates to methods and a device for obtaining biological samples from nipples in mammalian females.

### BACKGROUND OF THE INVENTION

The continued increase in awareness of the physical and emotional benefits of breastfeeding for the mother and baby has helped to increase the number of mothers who initiate breastfeeding. These benefits include increased protection of the infant from illness through the development of protective antibodies, decreased risk of developing childhood cancers, avoiding potential allergies to commercial infant formulas, and enhanced jaw, teeth, and speech development. Furthermore, it has been suggested that nursing mothers have a lower risk of developing breast cancer. Breast feeding has also been suggested to improve the emotional bond between mother and child. The initiation rate for breastfeeding is somewhere between 70%-80% of new mothers. The healthcare community has also become much more aware of the benefits of breastfeeding and in recent years has become much more proactive in their efforts to help new mothers succeed in their breastfeeding experience.

Nipple/areola soreness, irritation, and damage are very common problems faced by new mothers worldwide after they give birth. It is demonstrated in clinical studies that between 85%-90% of new mothers deal with nipple/areola pain and soreness. Clinical tests demonstrate that many new mothers quit breastfeeding when confronted with these challenges due to the pain and health complications associated with these issues. Causes of nipple soreness, irritation, etc. include, improper positioning of the infant on the mothers breast when breastfeeding and wet saturated breast pads kept on the nipple for extended periods of time, as well as others. One major issue mothers face when breastfeeding is what to do at night time when the infant begins to sleep for longer periods of time and they continue to produce milk and saturate their breastpads and brassiere. It is a major issue for many mothers who have to wear a brassiere to bed at night in order to hold the pads or other apparatus' in place. Mothers are left with the option of either going without a brassiere and leaking all over themselves and their bed or wearing a brassiere and having wet saturated breast pads on their nipple area for extended periods of time which again leads to soreness, etc.

Another significant issue facing lactating mothers is breast infections. One of the most prevalent infections is mastitis, a bacterial infection that mothers experience after they give birth. Mastitis can be caused by plugged milk ducts and cracked nipples, for example. Topical medications and antibiotics are typically provided to help fight the infection.

A second infection faced by breastfeeding mothers is thrush. Thrush is a yeast infection (a fungus) of the infant mouth, which is then passed to the mother during breastfeeding. Thrush can occur when antibiotics are provided to the mother after giving birth, especially after a cesarean section.

Nipple soreness, irritation, damage, and infection require a great deal of time and effort by both the healthcare professional and the mother to treat effectively. The mother is also confronted with the cost of treating these problems, as well as living with the discomfort. Thus, healthcare professionals struggle to find sufficient solutions to help new mothers, whether they are breastfeeding or not, overcome these issues. New solutions are very important in the effort to promote a long and successful breastfeeding experience for as many new mothers as possible. The current methods available to healthcare professionals and new mothers all fall short of alleviating and/or remedying the issues of sore, irritated, damaged, or infected nipples/areolas.

Products currently designed to alleviate problems associated with nipple/areola soreness, irritation, damage or infection include breast pads, breast shells, leakage inhibitors, nipple creams, and hydrogel pads. Breast pads absorb leakage, however, the pads become saturated with fluid and can not absorb any excess. Thus, the nipple area remains wet and the saturated pad no longer performs to its intended use. It has been clinically documented that moisture of this kind on the skin covered by a wet pad can lead to tissue breakdown, fungal and bacterial growth which then leads to maceration, breast infections (e.g., mastitis), plugged ducts, abscesses, etc. The breast shell is designed to relieve sore nipples. However, the design of the breast shell leads to excessive amounts of leakage resulting in potential complications for the new mother. In addition, the breast shell does not offer any preventative measures and is very bulky under clothing, making it cosmetically unappealing. Leakage inhibitors are designed to prevent leakage. However, there is not a sufficient barrier between the plastic of the leakage inhibitor and the skin to help prevent or decrease soreness and/or keep the nipple/areola dry. In addition, the leakage inhibitors do not have the ability to transport preventive or healing agents of any kind. They also must be placed in a bra, thus decreasing their effectiveness at night. Nipple creams are designed to heal sore nipples. However, again, there is no barrier between the nipple and the pad, bra, or clothing, which leaves the nipple susceptible to rubbing and abrasions. It is also messy to apply and remove. Hydrogel pads are designed to provide a moist healing environment and barrier between the nipple and bra/clothing and they absorb moisture/leakage from or around the nipple. Some gel pads also claim to have bacteriostatic and fungistatic properties that help to heal sore or macerated nipples. However, the hydrogel pad only absorbs a small amount of moisture/fluids (colostrum, leaking milk, etc.). The pad becomes saturated very quickly and becomes ineffective and must be discarded. It also loses its bacteriostatic and fungistatic properties when saturated. Again, much like the breast pad, the moist environment created by the gel pad can lead to tissue breakdown, bacterial growth, etc.

Another problem faced by women, including pregnant and lactating mothers, is detection and identification of breast infections (as described above), as well as breast diseases, such as cancer. Breast cancer is a significant cause of death in women. In particular, breast cancer is often discovered at a late stage of the disease, when therapeutic options and survival rates have dropped. In addition to the problem of early detection, there remain serious problems in distinguishing between malignant and benign breast disease, in staging known breast cancers, and in differentiating between different types of breast cancers. Recent efforts to develop improved methods for breast cancer detection, staging, and classification have focused on a promising array of cancer “markers.” Cancer markers are typically proteins that are uniquely expressed by cancerous cells, or are expressed at measurably increased or decreased levels by cancerous cells as compared to normal cells. Other cancer markers can include specific DNA or RNA sequences marking deleterious genetic changes in patterns or levels of gene expression associated with particular forms of cancer. A large number and variety of breast cancer markers have been identified to date, and many of these have been shown to have important value for determining prognostic and/or treatment-related variables.

However, current techniques for obtaining a biological sample for testing in a breast cancer marker assay are often invasive in nature and, therefore, are undesirable. For example, current techniques include the use of conventional or needle biopsy samples.

Non-invasive techniques for obtaining a biological sample have been developed and include breast serum analyses, studies of mammary fluid obtained from patients presenting with spontaneous nipple discharge, and the use of breast pumps in combination with oxytocin administration to study mammary fluid (see U.S. Pat. Nos. 5,798,266 and 6,287,521, both to Quay et al.). However, breast serum analyses have met with limited success, largely because the targeted markers are either not detectable in serum, or because telltale changes in the levels or activity of the markers cannot be monitored in serum. Moreover, the studies of mammary fluid from patients presenting with spontaneous nipple discharge is limited to the rare condition of spontaneous nipple discharge. Further, the use of breast pumps in combination with oxytocin administration is not suitable for pregnant mothers, as this combination stimulates lactation, hormone production, and uterine contractions. In addition, the use of breast pumps in combination with oxytocin administration requires significant expenditures by the user for both the equipment (i.e., breast pump) and the drug therapy, as well as requiring a cumbersome process for collecting the sample.

Therefore, there continues to be a need for a device and method that effectively prevents and/or heals sore, irritated, damaged, or infected nipples/areolas, and also has the ability to deliver drugs or other agents to this area. In addition, there continues to be a need for an inexpensive and easy-to-use device and method for the detection of breast disease and/or infection in women. In particular, a device and method that can detect breast disease and/or infection during a mother's childbearing years, especially for pregnant or lactating women, before the disease and/or infection has a chance to occur, grow, or spread is needed.

### SUMMARY OF THE INVENTION

The present invention relates to a method for preventing or healing injured nipples or areolas in mammalian females. The method involves positioning an absorbent medium over at least a portion of a nipple and areolar region of a mammalian female breast. Next, pressure (pressure, as used herein, is intended to indicate positive pressure) is applied to the absorbent medium over at least a portion of the nipple and areolar region sufficient to prevent or heal injured nipples or areolas. As used herein, injured nipples or areolas include sore, irritated, damaged, or infected nipples or areolas.

The present invention also provides a method for preventing or healing injured nipples or areolas in mammalian females using a plurality of nipple/areola healing devices during a period of lactation. Each of the devices includes a base having a pressure region which is located on an interior surface of the base. The pressure region includes an absorbent medium. The method involves positioning one of the devices over a mammalian female breast such that the pressure region is positioned over at least a portion of a nipple and areolar region of the breast. Next, pressure is applied on the one of the devices such that the pressure region applies pressure to at least a portion of the nipple and areolar region sufficient to prevent or heal injured nipples or areolas. Subsequently, the one of the devices is disposed of. Next, another one of the devices is positioned over the breast such that the pressure region is positioned over at least a portion of the nipple and areolar region. Pressure is then applied on the another one of the devices such that the pressure region applies pressure to at least a portion of the nipple and areolar region sufficient to prevent or heal injured nipples or areolas. Subsequently, the another one of the devices is disposed of. The steps of positioning and disposing of the devices occur during the period of lactation.

Another aspect of the present invention relates to a device for preventing or healing injured nipples or areolas in mammalian females. The device comprises a base having an interior surface. The interior surface of the base includes a pressure region which includes an absorbent medium. The pressure region including the absorbent medium applies pressure to at least a portion of a nipple and areolar region of a mammalian female breast sufficient to prevent or heal injured nipples or areolas.

The device can be used alone or can be part of a system which includes a brassiere in which the device is either placed into or integrated with (e.g., sewn into). The brassiere can be either a conventional or breastfeeding brassiere.

Yet another aspect of the present invention relates to a method for obtaining a biological sample from a mammalian female nipple. This method involves positioning an absorbent medium over at least a portion of a nipple region of a mammalian female breast and applying pressure to the absorbent medium over at least a portion of the nipple region sufficient to obtain a biological sample on or within the absorbent medium.

A further aspect of the present invention relates to a device for obtaining a biological sample from a mammalian female nipple. The device comprises a base having an interior surface. The interior surface of the base includes a pressure region which includes a hollow cavity. The pressure region applies pressure to at least a portion of an areolar region of a mammalian female breast and the hollow cavity is positioned to obtain a biological sample within the hollow cavity.

The present invention provides a convenient and effective way to prevent or heal sore, irritated, damaged, or infected nipples or areolas. In particular, the use of an absorbent medium to absorb excess exudate, thereby reducing the moisture in the wound area, along with the pressure applied directly to the wounded nipple and/or areola, in conjunction with frequent changes of the absorbent medium reduces the level of bacteria in the wound site, thus stimulating the healing process and decreasing the risk of soreness, irritation, and infection. Moreover, the device can be inexpensively constructed in a variety of shapes from a variety of materials. In addition, the absorbent medium can be used to deliver medicinal agents, such bacteriostatic, fungistatic, and microbicide agents, or any pharmacological, chemical, natural, or homeopathic agents, to the nipple and areolar region.

Further, the present invention provides a non-invasive and preventative way to obtain and analyze a biological sample from a mammalian female, preferably, a human female. In particular, the biological sample may be analyzed for oncogenes, pre-cancerous DNA evidence, and other markers for disease, infection, or other conditions.

### BRIEF DESCRIPTION OF THE DRAWINGS

FIG. 1 is a cross-sectional side view of a system for preventing or healing injured nipples or areolas in mammalian females placed over a human female breast;

FIG. 2 is a front view of a base of the system shown in FIG. 1;

FIG. 3 is a cross-sectional side view of the base taken along line 3-3 in FIG. 2;

FIG. 4 is a front view of a second embodiment of the base of the present invention;

FIG. 5 is a cross-sectional side view of the base taken along line 5-5 in FIG. 4;

FIG. 6 is a front view of a third embodiment of the base of the present invention;

FIG. 7 is a cross-sectional side view of the base taken along line 7-7 in FIG. 6;

FIG. 8A is an exploded view and FIG. 8B is a perspective view of a fourth embodiment of a device in accordance with the present invention;

FIG. 9A is an exploded view and FIG. 9B is a perspective view of a fifth embodiment of a device in accordance with the present invention;

FIG. 10A is an exploded view and FIG. 10B is a perspective view of a sixth embodiment of a device in accordance with the present invention;

FIG. 11A is an exploded view and FIG. 11B is a perspective view of a seventh embodiment of a device in accordance with the present invention;

FIG. 12A is an exploded view and FIG. 12B is a perspective view of a eighth embodiment of a device in accordance with the present invention;

FIG. 13A is an exploded view and FIG. 13B is a perspective view of a ninth embodiment of a device in accordance with the present invention;

FIGS. 14A-B are perspective views of a tenth embodiment of a base of the present invention;

FIGS. 15A-B are perspective views of an eleventh embodiment of a base of the present invention;

FIGS. 16A-B are perspective views of a twelfth embodiment of a base of the present invention;

FIG. 17 is a perspective view of a thirteenth embodiment of a base of the present invention;

FIGS. 18A-B are perspective views of a fourteenth embodiment of a base of the present invention; and

FIG. 19A is a front view and FIG. 19B is a side view of a fifteenth embodiment of a base of the present invention;

### DETAILED DESCRIPTION OF THE INVENTION

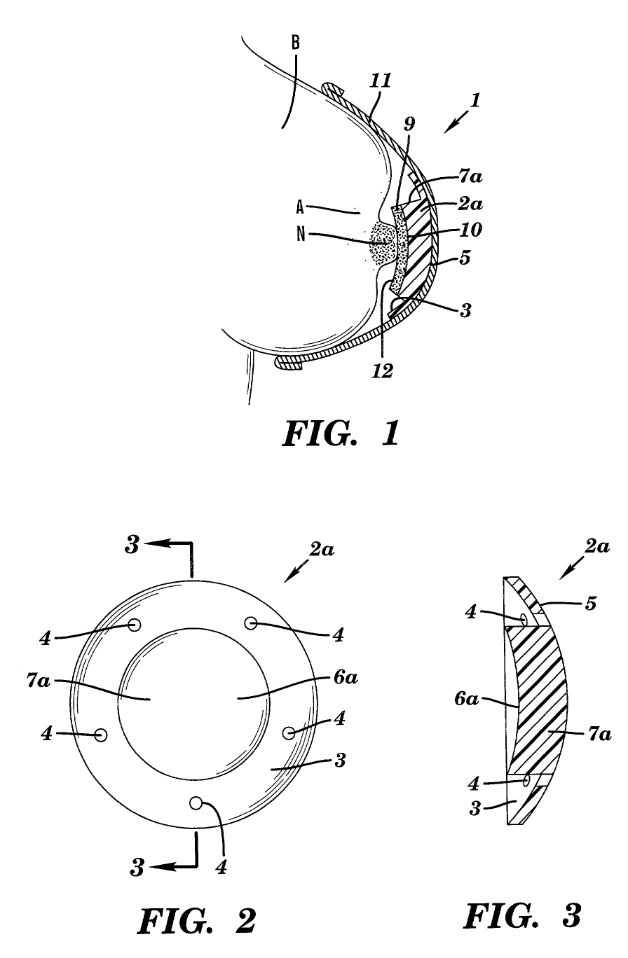

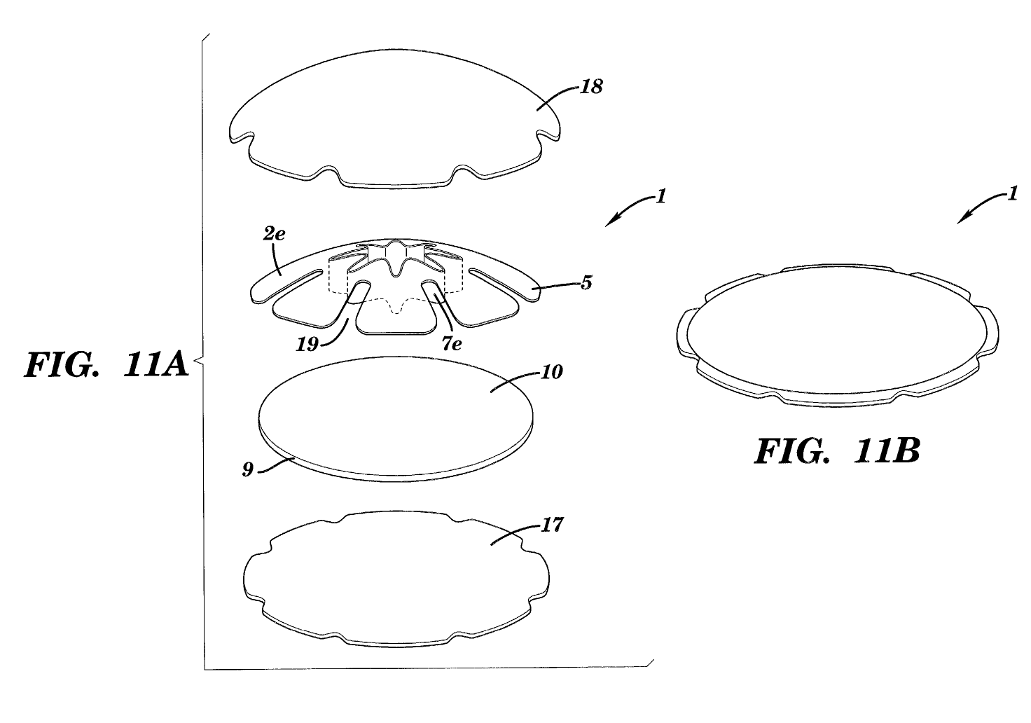

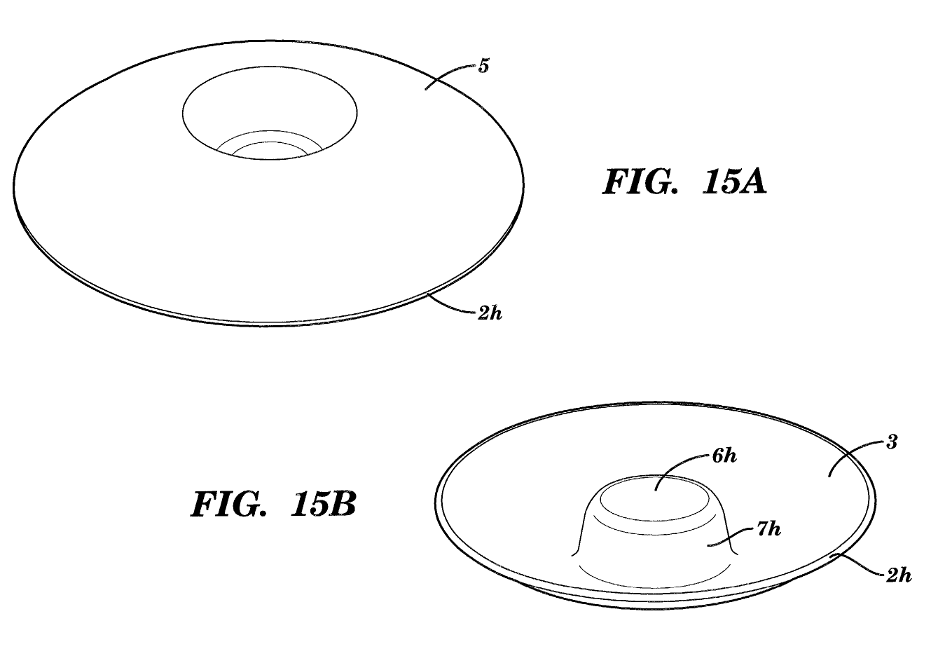

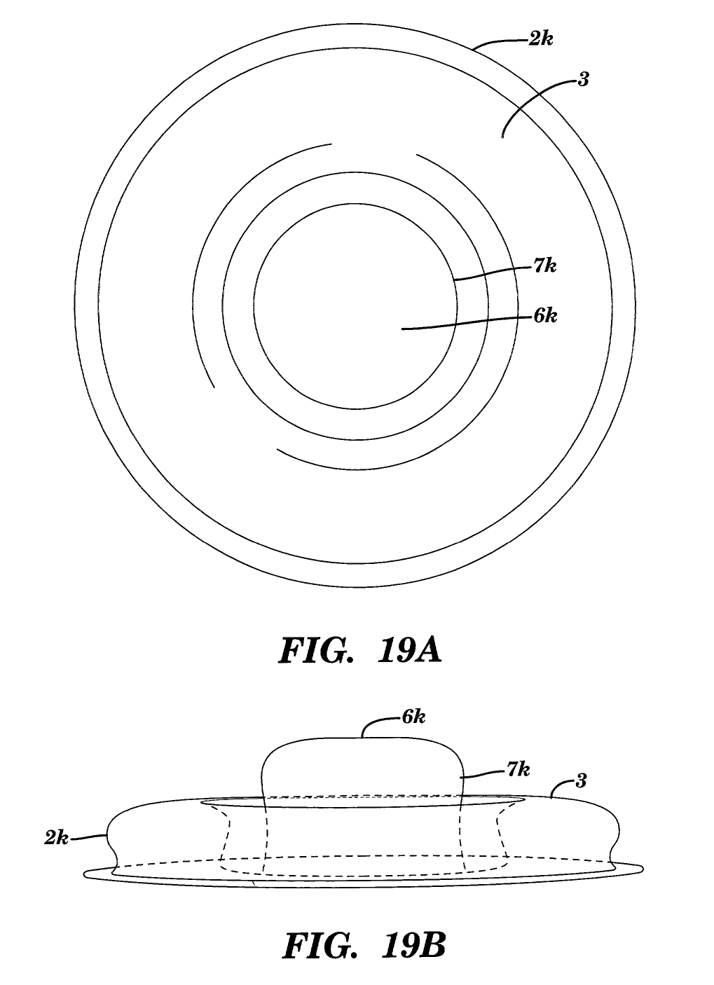

A device 1 for preventing or healing injured nipples and/or areolas in mammalian females is illustrated in FIGS. 1-19. The device in FIGS. 1-19 is particularly suited for preventing or healing injured nipples and/or areolas in human females. Therefore, the following description of FIGS. 1-19 is directed to preventing, healing, or obtaining samples from human females, however, the methods and devices disclosed herein are also useful for other mammals. The device includes a base 2a, 2b, 2c, 2d, 2e, 2f, 2g, 2h, 2i, 2j, or 2k having a region 7a, 7b, 7c, 7d, 7e, 7f, 7g, 7h, 7i, 7j, or 7k which places pressure on at least a portion of the nipple and areolar region of the breast. The pressure region 7a, 7b, 7c, 7d, 7e, 7f, 7g, 7h, 7i, 7j, or 7k includes an absorbent medium 9. The combination of direct, localized pressure and an absorbent medium prevents injury to the nipple and promotes the healing process. In particular, the direct, localized pressure applied through the pressure region decreases or eliminates fluid release from the nipple/areola, which allows the absorbent medium to more effectively function to absorb any fluid in the nipple and areolar region. In addition, the direct delivery of medicinal agents to the wounded area through the absorbent medium accelerates the healing process.

Referring to FIGS. 1-3, device 1 includes base 2a having an interior surface 3 and an exterior surface 5. In this particular embodiment, the interior surface 3 has a pressure region or projection 7a which extends away from base 2a. The interior surface 3 defines a nipple-contacting region 6a at projection 7a. Projection 7a is positioned to align substantially with nipple N and areola A (the nipple and areolar region) of human female breast B. Projection 7a operates to depress nipple N and areola A, whereby soreness, irritability, damage, and infection of nipple N and areola A is prevented or healed. However, any mechanism for applying pressure to the nipple N and areola A is suitable.

In the present invention, the device 1 includes an absorbent medium 9, as shown in FIG. 1. In this particular embodiment, the absorbent medium 9 is adjacent and in contact with the nipple-contacting region 6a of the interior surface 3 of the base 2a at the location of the projection 7a. The absorbent medium 9, in this embodiment, has an outer surface 10 and an inner surface 12 and at least a portion of the outer surface 10 is in contact with nipple-contacting region 6a of the interior surface 3 at the location of projection 7a. However, the absorbent medium 9 may be located at different positions with regard to the base 2a or may integrated with the interior surface 3 of the base 2a. In one embodiment, the projection 7a may be formed of the absorbent medium 9.

An attachment device may be provided which attaches the nipple-contacting region 6a of the interior surface 3 to at least a portion of the outer surface 10. Suitable attachment devices are known in the art and include, but are not limited to, adhesive tape, Velcro™, liquid adhesive material, compression, bonding agents (e.g., glue), and heat seals. The absorbent medium 9 may provide soothing/cooling properties and can absorb moisture on and around the nipple and areolar region. Absorbent medium 9 has a substantially circular shape, with a cross-sectional area which is the same as or larger than the size of a human female nipple and/or areola, although the shape and size of the absorbent medium 9 can vary as desired. Suitable absorbent materials include, but are not limited to, cotton, tissue paper, and hydrogels.

In one embodiment of the present invention, the absorbent medium 9 further includes a medicinal agent. In particular, a medicinal agent may be applied on the surface of or within the absorbent medium 9. Suitable medicinal agents include bacteriostatic, fungistatic, and microbicide agents, or any pharmacological, chemical, natural, or homeopathic agents. Since the pressure region or projection 7a, 7b, 7c, 7d, 7e, 7f, 7g, 7h, 7i, 7j, or 7k in contact with the absorbent medium 9 applies pressure to the nipple and areola region, the medicinal agent is delivered to the nipple and areola region through the absorbent medium.

Referring to FIGS. 1-3, base 2a is shaped to conform substantially to a human female breast. In this embodiment, base 2a is constructed in a substantially circular form with a concave interior surface 3 and a convex exterior surface 5. However, the base 2a may take other forms, including a flat interior and exterior surface. In this particular embodiment, base 2a covers a relatively small area of breast B as shown in FIG. 1 and can be used on breasts of various sizes. In another embodiment, base 2a is shaped such that suction is created between breast B and device 1 after device 1 is placed over breast B. The suction helps to maintain the alignment of device 1 with nipple N and/or areola A.

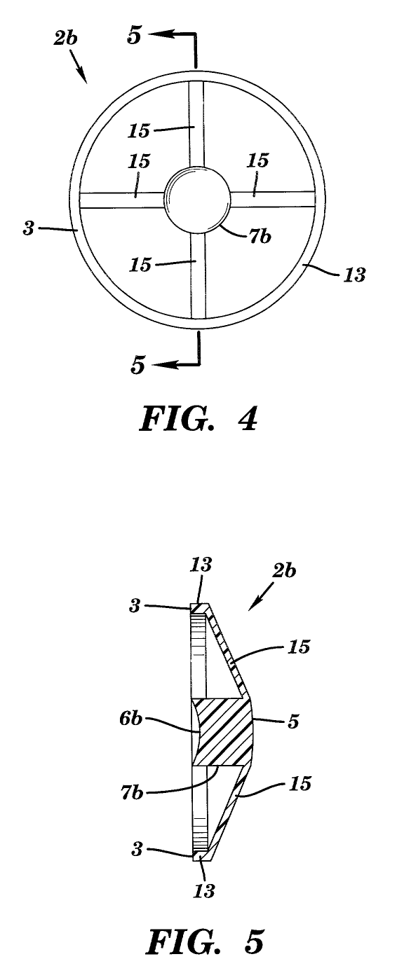

Base 2 can also take a variety of other forms, substantially conforming to larger or smaller areas of breast B. For example, another embodiment of a base 2 in accordance with the present invention is shown in FIGS. 4-5. In this embodiment, the base 2b includes an outer rim 13 connected to the projection 7b (having nipple-contacting region 6b) with a plurality of arms 15 to produce a very lightweight device.

Base 2b, in this particular embodiment, is made from a disposable material, such as paper (e.g., molded paper fiber, which can be made, for example, using a screen form or by pressing), cardboard (e.g., corrugated or non-corrugated cardboard, which may be die cut), cotton, or lightweight, inexpensive plastic (e.g., injection molded polypropylene, starch plastic resin, polypropylene sheet, or polyethylene sheet, which may be vacuum formed), although other materials may be used. In this embodiment, the device 1 is used for a short period of time and changed frequently, thus decreasing the chance for soreness, maceration, or bacterial and/or fungal growth and improving the healing properties of the device. However, the base 2b can be made from a variety of materials. Suitable other materials include flexible materials, such as moldable plastic or rubber. The use of a moldable, flexible plastic material allows user comfort and potentially ease of manufacture.

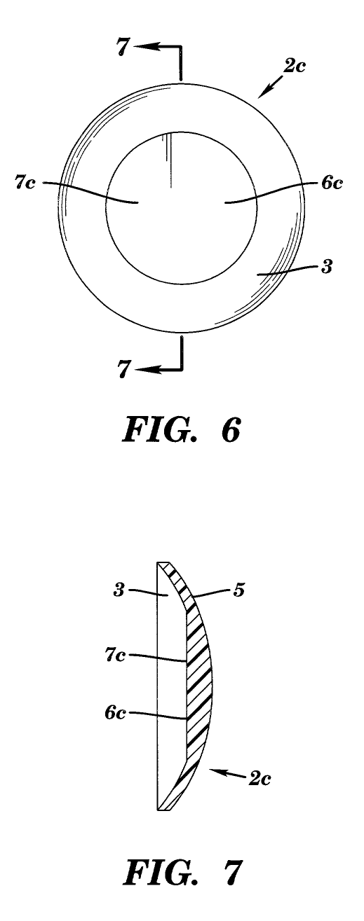

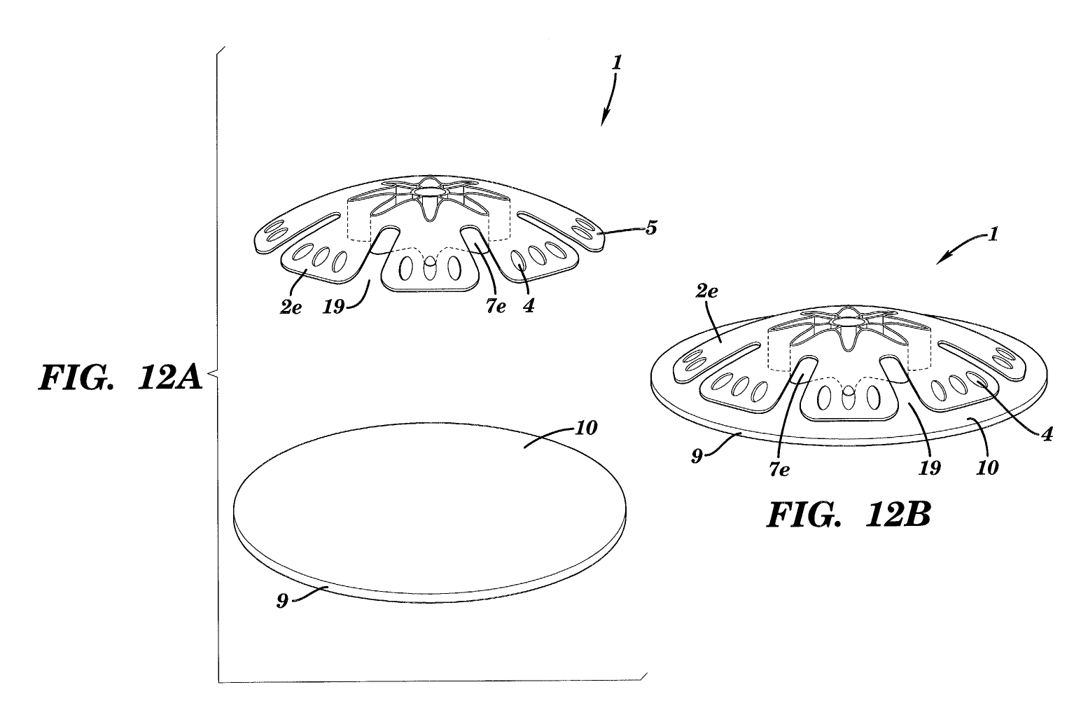

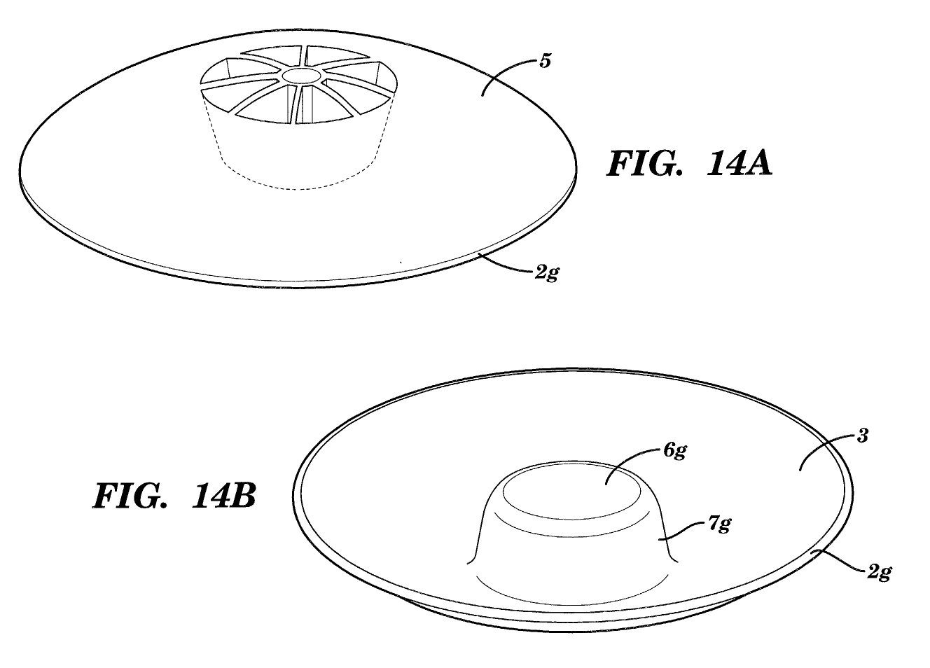

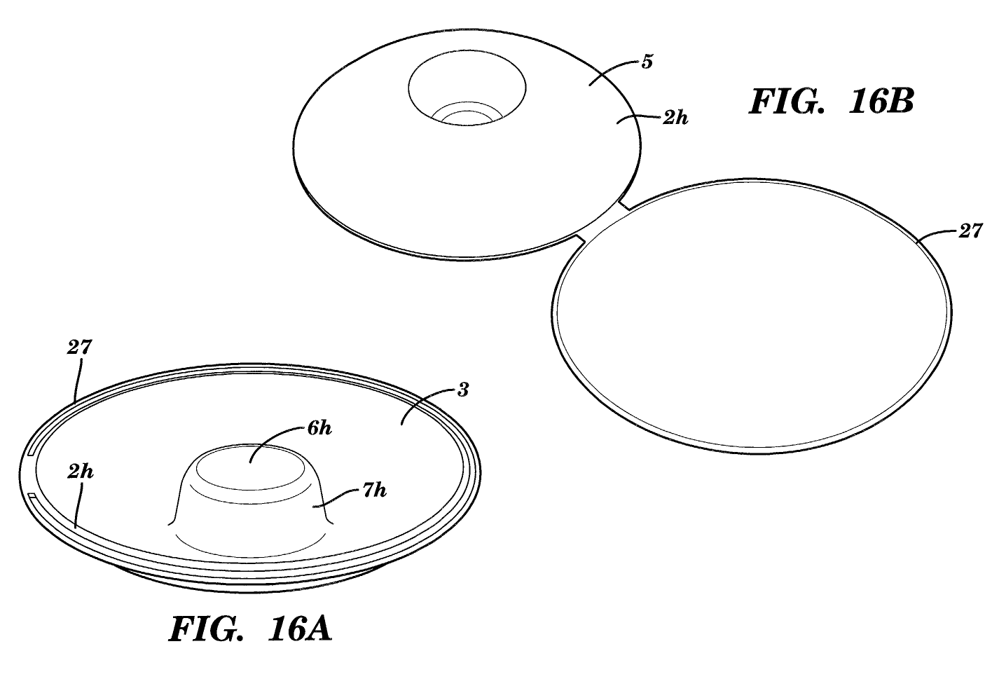

Yet further embodiments of base 2 in accordance with the present invention are shown in FIGS. 6-19. For example, in FIGS. 14A-B, the base 2g is designed to minimize the open area on the outside while maximizing the pressure area. Base 2h in FIGS. 16A-B includes an outer layer 27 hinged to the base 2h, which creates a shape that is smooth on the outside. Further, base 2k in FIGS. 12A-B includes a flexible material, such as low density polypropylene sheet, which is sealed and inflated.

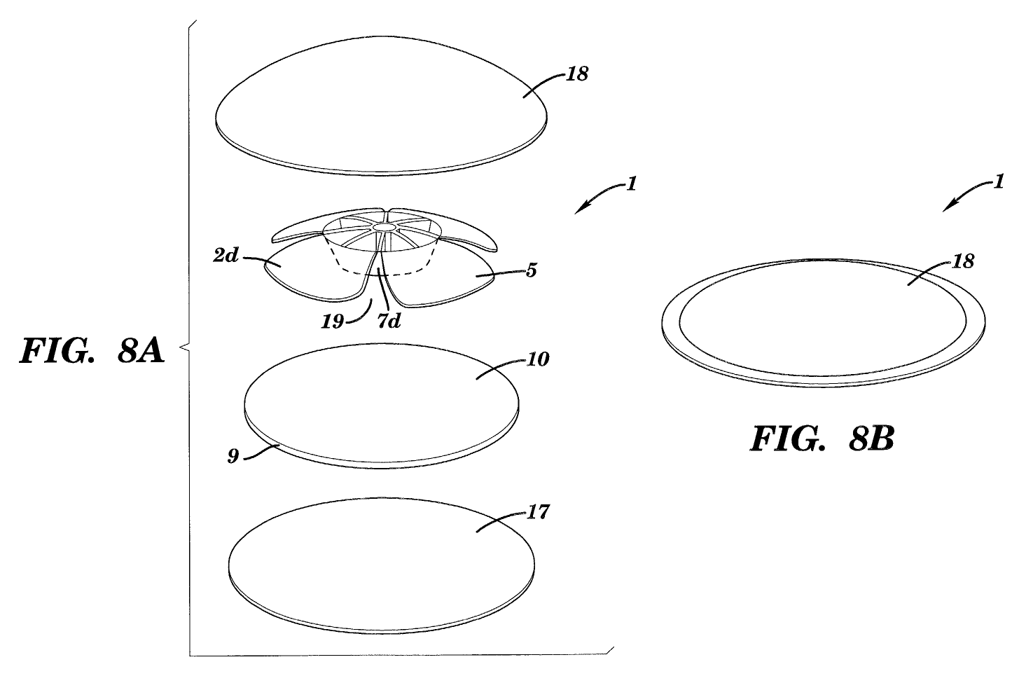

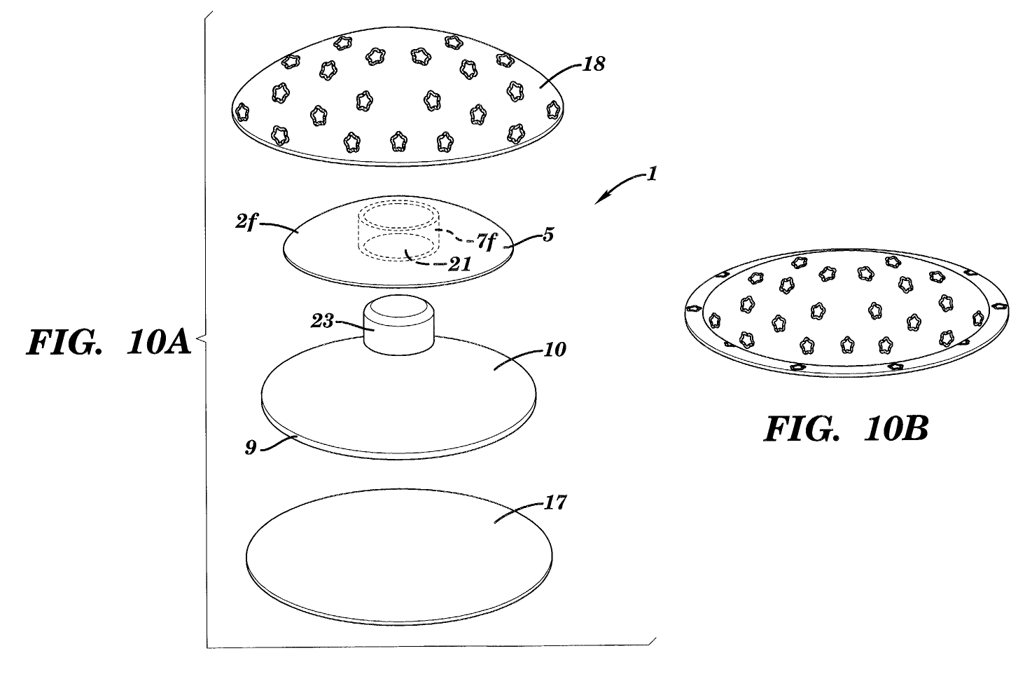

Referring to FIGS. 8-11 and 13, the device 1 may include a comfort layer 17 adjacent the inner surface 12 of the absorbent medium 9, thus improving the feel of the device when placed against the user's skin. Suitable materials for the comfort layer 17 include cotton, paper (e.g., tissue paper), and other lightweight, aesthetically pleasing materials. Again, referring to FIGS. 8-11 and 13, the device 1 may also include a cosmetic layer 18 adjacent the exterior surface 5 of the base. Suitable materials for the cosmetic layer 18 include cotton, paper (e.g., tissue paper), and other lightweight, aesthetically pleasing materials. The cosmetic layer 18 may include, for example, printed patterns (see FIGS. 10A-B) or embossed patterns. When both a comfort layer 17 and a cosmetic layer 18 are used, the comfort layer 17 and the cosmetic layer 18 may be sealed, e.g., by heat sealing, to each other around their perimeters to enclose the base 2 and absorbent medium 9 (see FIGS. 8B, 9B, 10B, 11B, and 12B).

Referring to FIGS. 2-3 and 12A, base 2a or 2e may also be provided with holes (or spaces) 4 to allow air circulation around the nipple and areolar region of breast B to further help prevent and heal local irritation. The number and placement of holes (or spaces) 4 may vary. As shown in FIGS. 8-9, 11A, 12A-B, and 17, base 2d and 2e may further be provided with a plurality of channels 19 which may be used alone or in combination with holes 4 to allow air circulation around the nipple and areolar region of breast B. The channels 19 also provide a flexible base 2 which can be used on breasts B of different sizes.

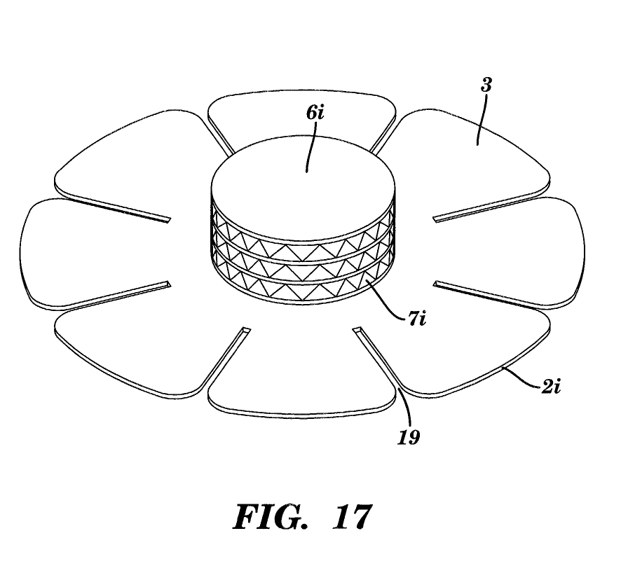

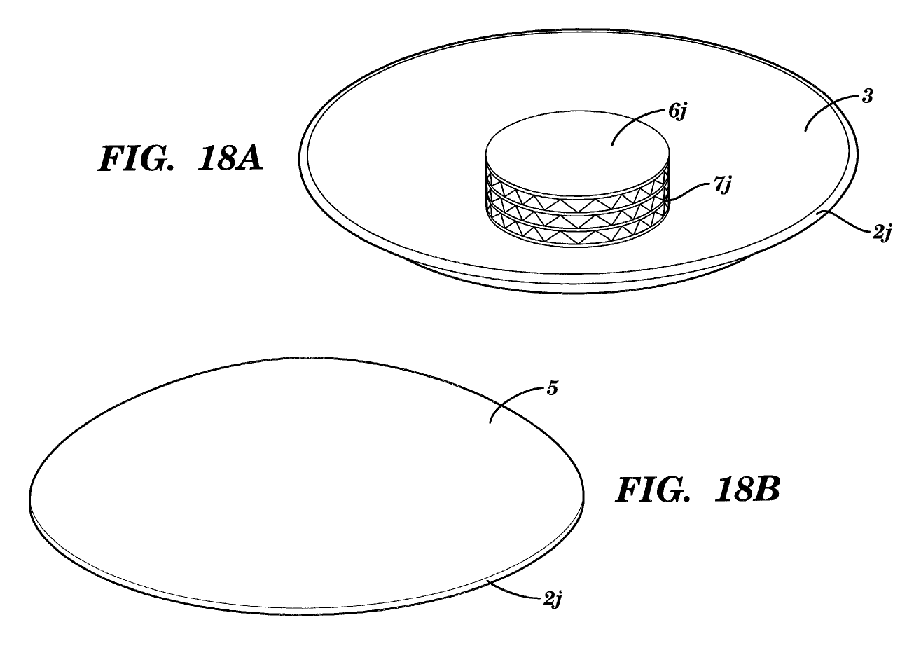

In the embodiment shown in FIGS. 1-3, projection 7a is integrated with the interior surface 3. Projection 7a is integrated with the interior surface 3 by forming projection 7a and base 2a together from the same material. Alternatively, projection 7a can be produced separately and then attached to interior surface 3 (see, e.g., FIGS. 17 and 18A). In this embodiment, projection 7a and base 2b can be made from the same or different materials. Projection 7a can be made from a variety of materials, as long as the material is sufficiently rigid to depress nipple N and/or areola A when nipple N and/or areola A is contacted by projection 7a. In this particular example, projection 7a is made from compressed paper. Other exemplary materials for forming projection 7a include flexible plastic, cardboard (e.g., corrugated or non-corrugated), and any of the rigid plastics known in the art or sufficiently rigidified rubber.

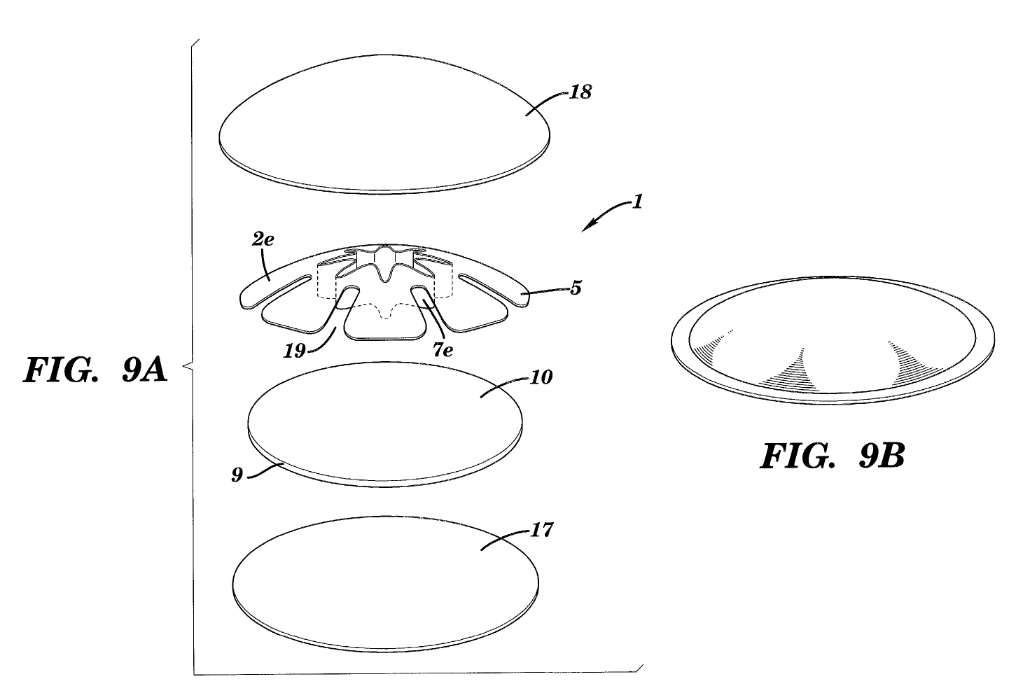

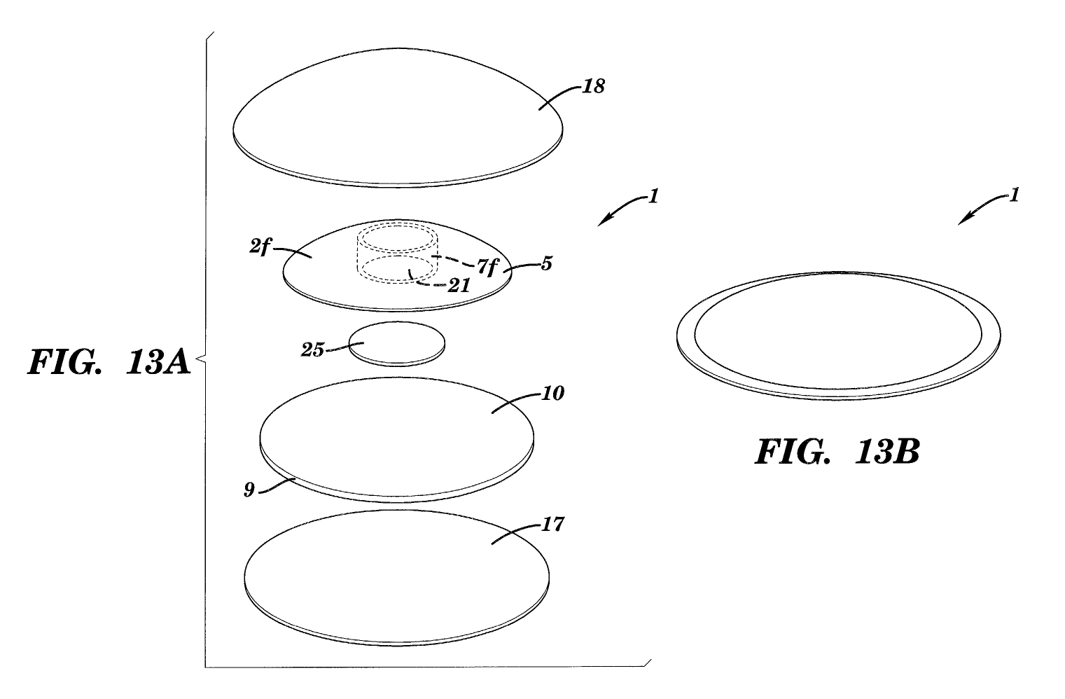

Referring to FIGS. 1-3, projection 7a is cylindrical in this example, having a size approximating a human female nipple and areolar region. However, other sizes and shapes may be used for the pressure region. In particular, projection 7a can be any shape, so long as it is capable of depressing nipple N and/or areola A when device 1 is brought into contact with breast B and, in turn, preventing or healing injured nipples or areolas. For example, an additional embodiment of the pressure region is shown in FIGS. 6-7. In this additional embodiment, the pressure region is a flattened, planar surface 7c formed in the center of base 2c which forms nipple-contacting region 6c of interior surface 3. Another embodiment of the pressure region in shown in FIGS. 9A, 11A, and 12A-B. In this embodiment, the pressure region is a star-shaped projection 7e formed in the center of base 2e having a size approximating a human female nipple and areolar region. Yet another embodiment of the pressure region is shown in FIGS. 10A and 13A. In this embodiment, the pressure region is an open cylinder 7f having a hollow cavity 21. As shown in FIG. 10A, in one embodiment, the hollow cavity 21 may house an absorbent wadding 23. The absorbent wadding 23 may be the same or a different material than the absorbent medium 9. Alternatively, as shown in FIG. 13A, the open cylinder 7f may include a substantially planar nipple-contacting plate 25 which is positioned adjacent the nipple-contacting surface of the open cylinder 7f to apply pressure over a larger portion of the nipple and areolar region.

As shown by FIG. 1, the above-described device can be used by placing it over breast B and applying pressure to the device such that the projection and the absorbent medium applies pressure to at least a portion of the nipple and areolar region sufficient to depress nipple N and areola A and, in turn, prevent or heal soreness, irritation, damage, or infection of nipple N and/or areola A of breast B. The amount of pressure need not be great and can normally be produced by the force provided when device 1 further comprises brassiere 11. Base 2a, 2b, 2c, 2d, 2e, 2f, 2g, 2h, 2i, 2j, or 2k can be placed inside the cup of brassiere 11 (with absorbent medium 9 adjacent and in contact with the interior surface 3 of base 2a, 2b, 2c, 2d, 2e, 2f, 2g, 2h, 2i, 2j, or 2k) which is then put on by the lactating woman, as illustrated by FIG. 1. Base 2a, 2b, 2c, 2d, 2e, 2f, 2g, 2h, 2i, 2j, or 2k can either be manually placed into or actually integrated with (e.g., sewn into) the cup of the brassiere. Brassiere 11 can be a conventional or nursing brassiere commonly worn by nursing mothers.

Alternatively, the device 1 may include an adhesive area, for example on at least a portion of the interior surface 3, which allows it to be placed on the nipple area without the need for a brassiere 11 or other apparatus to keep it in place.

In accordance with the present invention, the device 1 of the present invention may be used in a method which uses a plurality of areola/nipple healing devices over a period of lactation. In particular, the device 1 is positioned such that the projection 7a, 7b, 7c, 7d, 7e, 7f, 7g, 7h, 7i, 7j, or 7k and absorbent medium 9 are positioned over the nipple N and areolar A region and pressure is applied on the device. The absorbent medium 9 is then able to exert its absorptive properties by absorbing moisture in the nipple N and areolar A region while simultaneously applying pressure to the nipple N and areolar A region through the projection 7a, 7b, 7c, 7d, 7e, 7f, 7g, 7h, 7i, 7j, or 7k to decrease or eliminate fluid release from the injured nipple/areola, thereby healing the injured nipple/areola. In addition, the absorbent medium 9 is positioned to deliver medicinal agents directly to the injured nipple or areola, thus reducing or eliminating the presence of bacteria and fungi and healing damage (e.g., cuts) to the nipple and areolar region. Subsequently, the device 1 is removed and disposed of and another device 1 in accordance with the present invention is positioned over the breast such that the projection 7a, 7b, 7c, 7d, 7e, 7f, 7g, 7h, 7i, 7j, or 7k and absorbent medium 9 is positioned over the nipple N and areolar A region. Pressure is then applied to the second device 1 to apply pressure to the nipple N and areolar A region a second time and, if present, to deliver a second portion of a medicinal agent to the nipple N and areolar A region. Subsequently, the second device 1 is removed and disposed of. The steps of positioning and disposing of the devices occur during the period of lactation. In addition, the steps of the method may be repeated with additional devices in accordance with the present invention, while the period of lactation continues or until soreness or infection is no longer an issue. In accordance with this embodiment of the present invention, a disposable device is typically used. Alternatively, in the above-described method, the absorbent medium 9 may be removed and disposed of and the base 2a, 2b, 2c, 2d, 2e, 2f, 2g, 2h, 2i, 2j, or 2k of the device 1 may be reused.

In accordance with another embodiment of the present invention, the device of the present invention may be used in a method for obtaining a biological sample from a mammalian female nipple. One method involves positioning an absorbent medium 9 over at least a portion of a nipple N of a mammalian female breast and applying pressure to the absorbent medium 9 over at least a portion of the nipple N sufficient to obtain a biological sample on or within the absorbent medium 9. The biological sample may be any secreted, discharged, or sloughed material from the nipple N. Suitable biological samples include, but are not limited to, breast fluid, colostrum, and milk. The biological sample may be collected in the form of whole fluid, whole cells or cellular components, other selected liquid or solid fractions of the fluid, purified or bulk proteins, glycoproteins, peptides, nucleotides or other desired constituents of the fluid.

In conjunction with or subsequent to sample collection, the biological sample may be exposed to other agents, such as buffers, diluents, cross-linking agents, extraction or chromatographic media, denaturing agents, etc., to stabilize or otherwise prepare the sample for processing in a desired assay, as set forth in U.S. Pat. No. 6,287,521, which is hereby incorporated by reference in its entirety.

The method for obtaining a biological sample may further include extracting the biological sample from the absorbent medium 9. Techniques for extracting the biological sample from the absorbent medium 9 are well known in the art and include, but are not limited to, applying pressure to the absorbent medium 9, e.g., by manually wringing the absorbent medium 9, under conditions effective to remove at least a portion of the biological sample from the absorbent medium 9.

Once extracted, the biological sample may be tested for indicators of disease, infection, or other conditions. Alternatively, the absorbent medium 9 including the biological sample may be used directly in an assay for indicators of disease, infection, or other conditions. In particular, the biological sample may be tested for the presence of markers for diseases such as breast cancer in a bioassay. Suitable markers include any cell, cell fragment, protein, peptide, glycoprotein, lipid, glycolipid, proteolipid, enzyme, DNA or RNA evidence (e.g., oncogenes and pre-cancerous DNA evidence), cytological features of whole cells present in the biological samples, and any other molecular or biological material associated with diseased or infected breasts. Markers in accordance with the present invention include, but are not limited to, markers of breast infections, benign neoplasia, malignant neoplasia, pre-cancerous conditions, and conditions associated with an increased risk of cancer. Breast disease markers are widely known and can be incorporated into the methods of the present invention. Suitable breast disease markers (including breast cancer markers) are described in U.S. Pat. No. 6,287,521; Liu et al., “Breast-Cancer Diagnosis with Nipple Fluid bFGF,” Lancet, 356(9229):567 (2000); Black et al., “The Diagnostic and Prognostic Utility of Prostate-Specific Antigen for Diseases of the Breast,” Breast Cancer Res. Treat., 59(1):1-14 (2000); Botsis et al., “Breast Cancer Markers During Normal Pregnancy,” Anticancer Research, 19(4C):3539-3541 (1999); Sauter et al., “Biological Markers of Risk in Nipple Aspirate Fluid are Associated with Residual Cancer and Tumour Size,” Br. J. Cancer, 81(7):1222-1277 (1999); Magklara et al., “Human Glandular Kallikrein in Breast Milk, Amniotic Fluid, and Breast Cyst Fluid,” Clin. Chem., 45(10):1774-1780 (1999), which are hereby incorporated by reference in their entirety. The presence or absence of gene or protein targets can provide clinicians with valuable information to gauge disease severity, stage, and aggressiveness. Suitable techniques for testing for indicators of disease, infection, or other conditions are well known and are set forth in U.S. Pat. No. 6,287,521, which is hereby incorporated by reference in its entirety. Such techniques include Enzyme Linked Immunosorbant Assay (ELISA) assays, immunoprecipitation assays, and various solid phase immunoassays, including Western blotting, dot blotting, and affinity purification immunoassays, as set forth in U.S. Pat. No. 6,287,521.

When a comfort layer 17 is used in the device of the present invention, the comfort layer 17 adjacent the inner surface 12 of the absorbent medium 9 may function as an additional absorbent layer. Thus, the biological sample may be extracted from the comfort layer 17 and/or the absorbent medium 9 or the comfort layer 17 may be used in an assay to test for indicators of disease, infection, or other conditions. Alternatively, if present, the comfort layer 17 adjacent the inner surface 12 may function as a filter, to differentially partition and absorb selected components of the biological sample. In this embodiment, the comfort layer 17 adjacent the inner surface 12 may remain dry against the skin, thus stimulating the healing process and decreasing the risk of soreness, irritation, and infection.

Yet another embodiment of the present invention relates to a device for obtaining a biological sample from a mammalian female nipple. Referring to FIGS. 10A and 13A, the device comprises a base 2f having an interior surface. The interior surface of the base includes a pressure region in the form of an open cylinder 7f having a hollow cavity 21. The pressure region applies pressure to at least a portion of an areolar region of a mammalian female breast and the hollow cavity 21 is positioned to obtain a biological sample within the hollow cavity 21. The biological sample may then be tested for markers for disease, infection, or other conditions, as described above.

The base 2f may include an absorbent wadding 23, which is placed within the hollow cavity 21 to collect the sample for testing. Alternatively, a test device may be placed within the hollow cavity 21. Suitable test devices include solid phase media for immobilizing a target marker in the biological sample, as described in U.S. Pat. No. 6,287,521, which is hereby incorporated by reference in its entirety. The sample or test device can then be subjected to a suitable bioassay, such as ELISA assays, immunoprecipitation assays, and various solid phase immunoassays, including Western blotting, dot blotting, and affinity purification immunoassays, as set forth in U.S. Pat. No. 6,287,521.

The device may be used in a method for obtaining a biological sample from a mammalian female nipple. This method involves positioning the device such that the pressure region 7f is positioned over a least a portion of the areolar region A of a mammalian female breast and applying pressure to the device sufficient to obtain a biological sample within the hollow cavity 21 of the pressure region. If an absorbent wadding 23 is present within the hollow cavity 21, the biological sample may then be extracted from the absorbent medium, if desired.

Another aspect of the present invention relates to kits for practicing the biological sample collection and assay methods of the present invention. The kits include a device for obtaining a biological sample in accordance with the present invention and a detecting device for detecting the presence and/or amount of a marker for breast infection, disease, or other conditions in the biological sample. Suitable detecting devices include reagents and other apparatuses, such as immunological and non-immunological probes for detecting the presence or amount of a breast cancer marker in the biological sample, as described in U.S. Pat. No. 6,287,521, which is hereby incorporated by reference in its entirety. The kits may also contain suitable buffers, preservatives such as protease inhibitors, direct or sandwich-type labels for labeling the probes, and/or developing reagents for detecting a signal from the label.

The methods and devices for obtaining a biological sample of the present invention allow a user to acquire a biological sample without the need for expensive equipment, such as a breast pump. The devices in accordance with the present invention are simple to use and, therefore, can be used at home without assistance by a medical professional. Further, the methods for obtaining a biological sample do not require creating a negative pressure surrounding the nipple using, for example, a breast pump. Moreover, the methods and devices in accordance with the present invention are safe for use by both pregnant and non-pregnant users. In contrast, the use of a breast pump in accordance with prior techniques stimulates lactation and hormone production which may be undesirable in a pregnant user. Further, the use of certain drugs, such as oxytocin, to stimulate expression of fluid from a nipple in prior art methods for obtaining a biological sample can stimulate uterine contractions, which may be unsafe for a pregnant user. Since the device and methods of the present invention are safe for both pregnant and non-pregnant users, they can be used, for example, to test for markers for cancer in pregnant users.

Although the invention has been described in detail, for the purpose of illustration, it is understood that such detail is for that purpose and variations can be made therein by those skilled in the art without departing from the spirit and scope of the invention which is defined by the following claims.

What is claimed is:

1. A method for preventing or healing injured nipples or areolas in mammalian females comprising:
positioning an absorbent medium over at least a portion of a nipple and areolar region of a mammalian female breast; and
applying pressure to the absorbent medium over at least a portion of the nipple and areolar region sufficient to prevent or heal injured nipples or areolas.

2. The method according to claim 1 wherein the absorbent medium is a gel pad, a cotton pad, or tissue paper.

3. The method according to claim 1 wherein the absorbent medium has bacteriostatic or fungistatic properties.

4. The method according to claim 1 wherein the absorbent medium is substantially the same size as the nipple and areolar region.

5. The method according to claim 1 wherein the absorbent medium is substantially larger than the nipple and areolar region.

6. The method according to claim 1 wherein applying pressure comprises positioning a pressure region of a device adjacent and in contact with the absorbent medium over at least a portion of the nipple and areolar region and placing the device within a brassiere.

7. The method according to claim 6 wherein the device is integrated with the brassiere.

8. The method according to claim 1 wherein applying pressure comprises adhering at least a portion of a device including a pressure region to the breast to position the pressure region adjacent and in contact with the absorbent medium over at least a portion of the nipple and areolar region.

9. The method according to claim 1 further comprising delivering a medicinal agent to the nipple and areolar region.

10. The method according to claim 9 wherein the delivering comprises applying the medicinal agent to the absorbent medium.

11. A method for preventing or healing injured nipples or areolas in mammalian females using a plurality of nipple/areola healing devices during a period of lactation, each of the devices including a base having an interior surface, wherein the interior surface of the base comprises a pressure region including an absorbent medium, the method comprising:
positioning one of the devices over a mammalian female breast such that the pressure region is positioned over at least a portion of a nipple and areolar region of the breast;
applying pressure on the one of the devices such that the pressure region applies pressure to at least a portion of the nipple and areolar region sufficient to prevent or heal injured nipples or areolas;
subsequently disposing of the one of the devices;
positioning another one of the devices over the breast such that the pressure region is positioned over at least a portion of the nipple and areolar region;
applying pressure on the another one of the devices such that the pressure region applies pressure to at least a portion of the nipple and areolar region sufficient to prevent or heal injured nipples or areolas; and
subsequently disposing of the another one of the devices, wherein the steps of positioning and disposing of the devices occur during the period of lactation.

12. The method according to claim 11 wherein the pressure region of the devices comprises a projection extending away from the base and positioned to align substantially with the nipple and areolar region.

13. The method according to claim 11 wherein the base of the devices is constructed from a disposable material.

14. The method according to claim 11 wherein the absorbent medium is a gel pad, a cotton pad, or tissue paper attached to the pressure region in the devices.

15. The method according to claim 11 wherein the absorbent medium has bacteriostatic or fungistatic properties.

16. The method according to claim 11 further comprising delivering a medicinal agent to the nipple and areolar region.

17. The method according to claim 16 wherein the delivering comprises applying the medicinal agent to the absorbent medium of the devices.

18. The method according to claim 11 wherein the pressure region of the devices is substantially the same size as the nipple and areolar region.

19. The method according to claim 11 wherein the pressure region of the devices is substantially larger than the nipple and areolar region.

20. The method according to claim 11 wherein applying pressure comprises placing the device within a brassiere.

21. The method according to claim 20 wherein the base is integrated with the brassiere.

22. The method according to claim 11 wherein applying pressure comprises adhering at least a portion of the device to the breast to position the pressure region including the absorbent medium over at least a portion of the nipple and areolar region.

23. A device for preventing or healing injured nipples or areolas in mammalian females comprising:
a base having an interior surface, wherein the interior surface of the base comprises a pressure region including an absorbent medium which applies pressure to at least a portion of a nipple and areolar region of a mammalian female breast sufficient to prevent or heal injured nipples or areolas.

24. The device according to claim 23 wherein the pressure region comprises a projection extending away from the base and positioned to align substantially with the nipple and areolar region.

25. The device according to claim 23 wherein the base is constructed from a disposable material.

26. The device according to claim 23 wherein the absorbent medium is a gel pad, a cotton pad, or tissue paper attached to the pressure region.

27. The device according to claim 23 wherein the absorbent medium has bacteriostatic or fungistatic properties.

28. The device according to claim 23 wherein the absorbent medium further comprises a medicinal agent.

29. The device according to claim 23 wherein the pressure region is substantially the same size as the nipple and areolar region.

30. The device according to claim 23 wherein the pressure region is substantially larger than the nipple and areolar region.

31. A system for preventing or healing injured nipples or areolas in mammalian females comprising:
a base having an interior surface, wherein the interior surface of the base comprises a pressure region including an absorbent medium which applies pressure to at least a portion of a nipple and areolar region of a mammalian female breast sufficient to prevent or heal injured nipples or areolas and
a brassiere having a cup, wherein the base is placed inside the cup of the brassiere.

32. The system according to claim 31 wherein the pressure region comprises a projection extending away from the base and positioned to align substantially with the nipple and areolar region.

33. The system according to claim 31 wherein the base is constructed from a disposable material.

34. The system according to claim 31 wherein the absorbent medium is a gel pad, a cotton pad, or tissue paper attached to the pressure region.

35. The system according to claim 31 wherein the absorbent medium has bacteriostatic or fungistatic properties.

36. The system according to claim 31 wherein the absorbent medium further comprises a medicinal agent.

37. The system according to claim 31 wherein the pressure region is substantially the same size as the nipple and areolar region.

38. The system according to claim 31 wherein the pressure region is substantially larger than the nipple and areolar region.

39. The system according to claim 31 wherein the base is integrated with the brassiere.

40. The system according to claim 31 wherein the brassiere is a conventional brassiere.

41. The system according to claim 31 wherein the brassiere is a nursing brassiere.

42. A method for obtaining a biological sample from a mammalian female nipple comprising:
positioning an absorbent medium over at least a portion of a nipple region of a mammalian female breast; and
applying pressure to the absorbent medium over at least a portion of the nipple region sufficient to obtain a biological sample on or within the absorbent medium.

43. The method according to claim 42 further comprising extracting the biological sample from the absorbent medium.

44. The method according to claim 42 wherein the absorbent medium is a gel pad, a cotton pad, or tissue paper.

45. The method according to claim 42 wherein the absorbent medium has bacteriostatic or fungistatic properties.

46. The method according to claim 42 wherein the absorbent medium is substantially the same size as the nipple region.

47. The method according to claim 42 wherein the absorbent medium is substantially larger than the nipple region.

48. The method according to claim 42 wherein applying pressure comprises positioning a pressure region of a device adjacent and in contact with the absorbent medium over at least a portion of the nipple and areolar region and placing the device within a brassiere.

49. The method according to claim 48 wherein the device is integrated with the brassiere.

50. The method according to claim 42 wherein applying pressure comprises adhering at least a portion of a device including a pressure region to the breast to position the pressure region adjacent and in contact with the absorbent medium over at least a portion of the nipple and areolar region.

51. The method according to claim 42 wherein the biological sample is breast fluid, colostrum, or milk.

52. A device for obtaining a biological sample from a mammalian female nipple comprising:
a base having an interior surface, wherein the interior surface of the base comprises a pressure region which includes a hollow cavity and wherein the hollow cavity is positioned to obtain a biological sample from a nipple of a mammalian female breast within the hollow cavity.

53. The device according to claim 52 wherein the pressure region comprises a hollow projection extending away from the base and positioned to align substantially with the areolar region.

54. The device according to claim 52 wherein the base is constructed from a disposable material.

55. The device according to claim 52 further comprising:
an absorbent wadding within the hollow cavity.

56. The device according to claim 55 wherein the absorbent wadding is a gel pad, a cotton pad, or tissue paper.

57. The device according to claim 55 wherein the absorbent wadding has bacteriostatic or fungistatic properties.

58. The device according to claim 52 further comprising: a test device within the hollow cavity.

59. The device according to claim 52 wherein the pressure region is substantially the same size as the areolar region.

60. The device according to claim 52 wherein the pressure region is substantially larger than the areolar region.

61. A method for obtaining a biological sample from a mammalian female nipple comprising:
positioning a device according to claim 52 such that the pressure region is positioned over at least a portion of an areolar region of a mammalian female breast; and
applying pressure to the device sufficient to obtain a biological sample from a nipple within the hollow cavity of the pressure region.

62. A kit for testing a biological sample from a mammalian female nipple for a marker for breast infection or disease comprising:
a device according to claim 52 for obtaining a biological sample from a mammalian female nipple; and
a detecting device for detecting the presence or amount of a marker for breast infection or disease in the biological sample.
